(19) United States
(12) Patent Application Publication
(10) Pub. No.: US 2016/0166569 A1
(43) Pub. Date: Jun. 16, 2016

(54) METHOD FOR TREATING SKIN THICKENING

(71) Applicant: GALDERMA RESEARCH & DEVELOPMENT, Biot (FR)

(72) Inventor: Guy BOUVIER, Biot (FR)

(73) Assignee: Galderma Research and Development, Biot (FR)

(21) Appl. No.: 14/907,706

(22) PCT Filed: Jul. 28, 2014
(86) PCT No.: PCT/US14/48439

§ 371 Date: Jan. 26, 2016

Related U.S. Application Data
(60) Provisional application No. 61/858,885, filed on Jul. 26, 2013.

(51) Int. Cl. A61K 31/498; A61Q 19/08; A61Q 17/04; A61K 8/49

(52) U.S. Cl. CPC A61K 31/498; A61K 8/494; A61Q 19/08; A61Q 17/04

(57) ABSTRACT

Methods, compositions and products for treating or reducing skin thickening in a subject are described. Also described are methods of inhibiting a skin disorder induced by an UV-irradiation. The methods involve topically administering to the subject a composition containing an et adrenergic receptor agonist, such as brimonidine.

### CROSS-REFERENCE TO RELATED APPLICATIONS

This application claims priority from [].S. Provisional Patent Application No. 61/858,885, filed Jul. 26, 2013, the disclosure of each of which is hereby incorporated by reference herein in its entirety.

### BACKGROUND OF THE INVENTION

Skin thickening occurs through abnormal cell proliferation, which can be triggered by various causes, including but not limited to, UV-radiation. Epidermal Growth Factor Receptor, or EGFR (also referred to as ErbB1 and HER.1), is a cell surface protein, which is activated by binding of its specific ligands, such as epidermal growth factor and transforming growth factor a (TGFa), etc. The activation of EGFR causes cell proliferation, inhibition of cell death, and increased epidermal hyperplasia, i.e., the increase in number and size of normal cells in normal arrangement, When skin is stimulated by UV radiation, EGFR increases keratinocyte proliferation, suppresses apoptosis, and augments and accelerates epidermal hyperplasia, (T.B. El-Abaseri and L.A. Hansen, “EGFR Activation and Ultraviolet Light Induced Skin Carcinogenesis,” Journal of Biomedicine and Biotechnology, vol. 2007, Article ID 97939, 4 pages, 2007), When stimulated by UV radiation, keratinocyte is activated through an EGFR dependent process and begins to create an abundance of keratinocyte cells, When the skin is exposed to UV-irradiation, apoptosis is suppressed by the EGFR-dependent pathway, causing cells to live longer than usual and also to carry genetic mutations that may lead to disease formation. The suppression of apoptosis, along with keratinocyte proliferation, considerably increases skin thickness and risks of other skin disorders or conditions.

Epidermal hyperplasia can be partially attributed to an injury in the basal layer keratinocytes of the cell, Shortly after skin is exposed to UV radiation the keratinocytes begin to divide and multiply at a more rapid pace. The EGER-dependent process can trigger the increase in cell production in any type of cells, from regular cells, cells suffering from epidermal hyperplasia, to the increased production of cancer cells. Pharmacological inhibition of the UV-induced activation of EGFR in genetically initiated mouse skin tumorigenesis model suppresses tumorignesis and the activation of mitrogen-activated protein (MAP) kinases and phosphatidyl inosito1-3-kinase (PI3K)/AKT signaling pathways. (T. B. El-Abaseri and L. A. Hansen, 2007, supra).

The α adrenoceptor agonists have been used therapeutically for a number of conditions including hypertension, congestive heart failure, angina pectoris, spasticity, glaucoma, diarrhea, and for the suppression of opiate withdrawal symptoms (J. P. Heible and R. R. Ruffolo Therapeutic Applications of Agents Interacting with a-Adrenoceptors, p.180-206 in Progress in Basic and Clinical Pharmacology Vol. 8, P. Lomax and E.S. Vesell Ed., Karger, 1991). Published U.S. Patent Application US20050276830 discloses α2 adrenergic receptor agonists and their use for treating or preventing inflammatory skin disorders.

There is a need of methods and compositions that would effectively treat or inhibit the progression of skin thickening. The present invention relates to such improved methods and compositions.

### BRIEF SUMMARY OF THE INVENTION

Treatment with an a adrenergic receptor agonist, such as brimonidine, has resulted in a significant reduction of skin thickening or other skin disorders in mammals, such as mice exposed to UV radiation.

In one general aspect, embodiments of the present invention relate to a method of reducing or inhibiting the progression of skin thickening in a subject in need thereof, comprising topically administering to a skin area of the subject a topical composition comprising an effective amount of at least one alpha adrenergic receptor agonist and a pharmaceutically acceptable carrier, wherein the skin area has, or is prone to have, skin thickening.

In a preferred embodiment, the skin thickening is induced by an UV-irradiation, such as by sun exposure. More preferably, the alpha adrenergic receptor agonist is biitnonidine.

In another general aspect, embodiments of the present invention relate to a method of inhibiting a skin disorder induced by an UV-irradiation in a subject in need thereof, comprising topically administering to a skin area of the subject a topical composition comprising an effective amount of at least one alpha adrenergic receptor agonist and a pharmaceutically acceptable carrier. In a preferred embodiment, the skin disorder, such as low grade, e.g., grade 1, of erythema or flaking, wrinkling or white raised area on skin, or skin thickening, is induced by sun exposure. More preferably, the alpha adrenergic receptor agonist is brimonidine.

In another general aspect, embodiments of the present invention relate to a method of regulating an EGFR response in a subject in need thereof to thereby result in treatment of a disease or condition associated with EGFR in the subject. The method comprises administering to the subject a composition comprising an effective amount of an a adrenergic receptor agonist and a pharmaceutically acceptable carrier.

Other aspects, features and advantages of the invention will be apparent from the following disclosure, including the detailed description of the invention and its preferred embodiments and the appended claims,

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set in the specification. All patents, published patent applications and publications cited herein are incorporated by reference as if set forth fuUy herein. It must be noted that as used herein and in the appended claims, the singular forms “a,” “an,” and “the” include plural reference unless the context clearly dictates otherwise.

As used herein, an “a adrenergic receptor agonist” or “agonist of et adrenoceptor” means a compound that binds to and stimulates alpha adrenergic receptor. An “a adrenergic receptor agonist” can be selective for an al adrenergic receptor, selective for an a2 adrenergic receptor, or nonselective for both an al adrenergic receptor and an α2 adrenergic receptor.

As used herein, the name of a compound is intended to encompass all possible existing isomeric forms (e.g., optical isomer, enantiomer, diastereomer, racemate or racemic mixture), esters, prodrugs, metabolite forms, or pharmaceutically acceptable salts, of the compound. For example, “brimonidine” can be the compound (5-bromo-quinoxalin-6-yl)-(4,5-dihydro-1H-imidazol-2-yl)-amine, and any pharmaceutically acceptable salt of the compound, such as brimonidine tartrate.

in an embodiment of the present invention, the a adrenergic receptor agonists include, but are not limited to, the a adrenergic receptor agonists disclosed in the published U.S. Patent Application US20050276830, which is herein incorporated by reference in its entirety.

Representative a adrenergic receptor agonists that can be used in the present invention include, but are not limited to, those listed in Table 1.

TABLE 1
Representative α adrenergic receptor agonists

| Compound Formula | Compound Name |
|---|---|
| 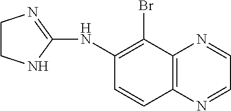 [SMILES: Brc1c(NC2=NCCN2)ccc2nccnc12] | (5-Bromo-quinoxalin-6-yl)-(4,5-dihydro-1H- imidazol-2-yl)-amine(Brimonidine) |
| 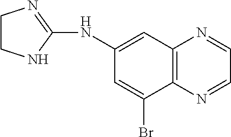 [SMILES: Brc1cc(NC2=NCCN2)cc2nccnc12] | (8-Bromo-quinoxalin-6-yl)-(4,5-dihydro-1H- imidazol-2-yl)-amine |
| 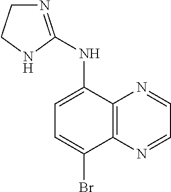 [SMILES: Brc1ccc(NC2=NCCN2)c2nccnc12] | (8-Bromo-quinoxalin-5-yl)-(4,5-dihydro-1H- imidazol-2-yl)-amine |
| 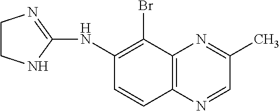 [SMILES: Cc1cnc2ccc(NC3=NCCN3)c(Br)c2n1] | (5-Bromo-3-methyl-quinoxalin-6-yl)-(4,5- dihydro-1H-imidazol-2-yl)-amine |
| 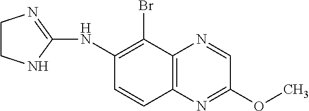 [SMILES: COc1cnc2c(Br)c(NC3=NCCN3)ccc2n1] | (5-Bromo-2-methoxy-quinoxalin-6-yl)-(4,5- dihydro-1H-imidazol-2-yl)-amine |
| 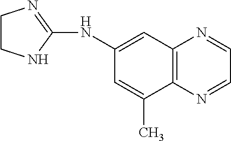 [SMILES: Cc1cc(NC2=NCCN2)cc2nccnc12] | (4,5-dihydro-1H-imidazol-2-yl)-(8-methyl- quinoxalin-6-yl)-amine |
| 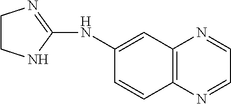 [SMILES: c1cnc2cc(NC3=NCCN3)ccc2n1] | (4,5-dihydro-1H-imidazol-2-yl)-quinoxalin-5-yl- amine |
| 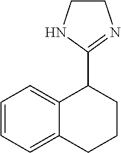 [SMILES: c1ccc2c(c1)CCCC2C1=NCCN1] | Tetrahydrozaline |
| 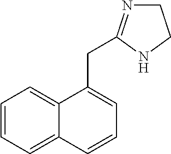 [SMILES: c1ccc2c(CC3=NCCN3)cccc2c1] | Naphazoline |
| 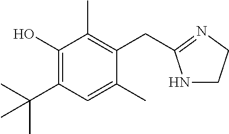 [SMILES: Cc1cc(C(C)(C)C)c(O)c(C)c1CC1=NCCN1] | Oxymetazoline |
| 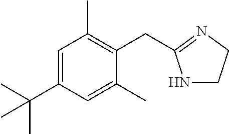 [SMILES: Cc1cc(C(C)(C)C)cc(C)c1CC1=NCCN1] | Xylometazoline |
| 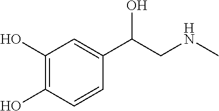 | Epinephrine |
| 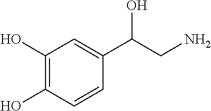 [SMILES: NCC(O)c1ccc(O)c(O)c1] | Norepinephrine |
| 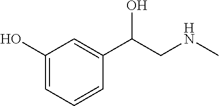 | Phenylephrine |
| 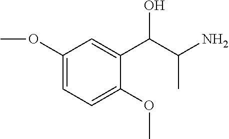 | Methoxyamine |

Preferably, thea adrenergic receptor agonist is an α2 adrenergic receptor agonist, most preferably brimonidine, (5-Bromo-quinoxalin-6-yl)-(4,5-dihydro-1H-imidazol-2-yl )-amine and pharmaceutically acceptable salts thereof, such as the tartrate salt of brimonidine.

Other examples of a adrenergic receptor agonists that can be used in the present invention include, but are not limited to, Dexmedetomidine, Medetomidine, Rotnifidine, Clonidine, Detomidine, Lofexidine, Xylazine, Tizanidine, Guanfacine, and Amitraz.

The phrase “pharmaceutically acceptable salt(s),” as used herein, means those salts of a compound of interest that are safe and effective for topical use in mammals and that possess the desired biological activity. Pharmaceutically acceptable salts include salts of acidic or basic groups present in the specified compounds. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicyl ate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds used in the present invention can form pharmaceutically acceptable salts with various amino acids. Suitable base salts include, but are not limited to, aluminum, calcium, lithium, magnesium, potassium, sodium, zinc, and diethanolamine salts. For a review on pharmaceutically acceptable salts see BERGE ET AL., 66 J. PHARR SCI. 1-19 (1977), incorporated herein by reference.

As used herein, the term “hydrate” means a compound of interest, or a pharmaceutically acceptable salt thereof that further includes a stoichiometric or non-stoichi metric amount of water bound to it by non-covalent intermolecular forces.

As used herein, the term “subject” means any mammal, preferably a human, to whom will be or has been administered compounds or formulations according to embodiments of the invention.

As used herein, the tertn “composition” is intended to encompass a product comprising the specified ingredients in the specified amounts, as well as any product which results, directly or indirectly, from combinations of the specified ingredients in the specified amounts.

As used herein, a “pharmaceutically-acceptable carrier” means a carrier that is pharmaceutically or cosmetically suitable for use in the present invention without causing undue or unacceptable toxicity, incompatibility, instability, irritation, allergic response, and the like. This term is not intended to limit the ingredient which it describes.

One general aspect of the present invention relates to a method of reducing or inhibiting the progression of skin thickening, preferably skin thickening induced by UV-irradiation, in a subject in need thereof. The method comprises topically administering to a skin area of the subject a topical composition comprising an effective amount of at least one alpha adrenergic receptor agonist and a pharmaceutically acceptable carrier, wherein the skin area has, or is prone to have, skin thickening.

Another general aspect of the present invention relates to a method of inhibiting or preventing another skin disorder induced by an UV-irradiation in a subject in need thereof, comprising topically administering to a skin area of the subject a topical composition comprising an effective amount of at least one alpha adrenergic receptor agonist and a pharmaceutically acceptable carrier.

As used herein, “a skin disorder induced by an UV-irradiation” refers to a skin disorder that occurs or develops resulting from the exposure of the skin to a UV radiation, excluding such skin disorder of a different etiology. Any skin disorder induced by UV-irradiation, including but not limited to, low grade, e.g., grade 1, of erythema or flaking, wrinkling or white raised area on skin, or skin thickening, can be inhibited or reduced by the present invention.

In one embodiment of the present invention, the topical composition is administered to the skin area before the UV-irradiation.

In another embodiment of the present invention, the topical composition is administered to the skin area after the UV-irradiation.

In yet another embodiment of the present invention, the topical composition is administered to the skin area before and after the UV-irradiation.

As used herein, “topical application,” “topical administration” or “topically applying” means direct application or administration onto skin or any other epithelium in need of treatment. According to embodiments of the present invention, a topical composition can be topically administered by directly laying or spreading on the skin or epithelium in need of the treatment, e.g., by use of the hands, an applicator or any other means.

As used herein, a “skin thickening” includes any abnormal increase in the number and/or volume of a cell or tissue in the skin due to UV radiation, either directly or indirectly. Preferably, the “skin thickening” is not associated with a skin tumor, which includes a skin cancer, a benign skin tumor and pre-malignant skin tumor, nor is “skin thickening” associated with rosacea, erythema, telangiectasias psoriasis, purpura, sagging skin or wrinkle. A “skin thickening” can be an abnormal increase in the number and/or volume of a cell or tissue in any layer of the skin, e.g., in the epidermis, dermis, and hypodermis. The cell or tissue in the skin can be, for example, keratinocytes, Merkel cells, melanocytes, Langerhans cells, fat cells, connective tissue, etc.

According to embodiments of the present invention, the “skin thickening” can also be associated with one or more conditions, such as that selected from the group consisting of sun exposure, hormonal imbalance, a deficiency in vitamin andlor antioxidant, epidermal hyperplasia, keratinocyte proliferation, EGFR-dependent cell division, and combinations thereof. As used herein, the term “skin thickening” encompasses epithelium hyperplasia, proliferation, pre-neoplasic transformation, in which EGFR may or may not have proven to play a key progression role. The term “skin thickening” also refers to all steps of cellular modifications leading epithelial thickening, and especially of the epidermis, excluding tumor formation. The “skin thickening” can be associated with one or more diseases or disorders, including, but not limited to, CREST syndrome, corns and calluses, warts, hives, keratosis, atopic dermatitis, eczema, scleroderma, lipoderatntoscelerosis, an age spot or lenti go.

As used herein, “hyperplasia” refers to the increased cell production in a normal tissue or organ. The term “hyperplasia” does not encompass tumor or cancerous changes of any skin cell.

One embodiment of the present invention relates to a method of preventing or inhibiting the progression of epidermal or epithelial hyperplasia in a subject, which comprises topically administering to the subject in need thereof a composition comprising an effective amount of an α adrenergic receptor agonist and a pharmaceutically acceptable carrier.

As used herein, pidermal or epithelial hyperplasia” refers to an abnormal increase in the number of cells, cell size and shape, in normal arrangement in organ or tissue, resulting in an increase in the organ or tissue volume. It can also be described as hypergenesis of the cells. Epidermal or epithelial hyperplasia can be triggered by anything from increased demand (i.e., to compensate for skin loss) to compensation for damage (i.e., an injury in the basal cell layer of skin or epithelium).

As used herein, “inhibit” or “inhibiting” refers to a reduction of the progression of skin thickening.

As used herein, an “effective amount of an a adrenergic receptor agonist” with respect to reducing or inhibiting the progression of skin thickening in a subject, means the amount of the a adrenergic receptor agonist that is sufficient to prevent or delay the progression of skin thickening in a subject.

Another general aspect of the present invention relates to a method of regulating an EGFR response in a subject to thereby result in treating or preventing of a disease or condition associated with EGFR in a subject, comprising administering to the subject in need thereof a composition comprising an effective amount of an a adrenergic receptor agonist and a pharmaceutically acceptable carrier.

As used herein, a “disease or condition associated with EGFR” can he any disease or condition that can be treated by regulating the activity of EGFR. Preferably, a “disease or condition associated with EGFR” is not associated with a skin tumor, which includes a skin cancer, a benign skin tumor and pre-malignant skin tumor, nor is the “disease or condition associated with EGER” associated with rosacea, erythema, telangiectasias psoriasis, purpura, sagging skin or wrinkle. Examples of “disease or condition associated with EGER” include non-skin tumors, such as tumors of the oral cavity, head and neck tissues, esophagus, including local and metastatic tumors located in these tissues; and other cell proliferative disorders, such as skin thickening. The term “disease or condition associated with EGFR” also encompasses hyperplasia, increased proliferation, and pre-neoplasic lesion.

According to the present invention, in a method of regulating an EGFR response in a subject, the a adrenergic receptor agonist can be administered to the subject through any route of administration, including, but not limited to topical, epicutaneous, transdermal, subcutaneous, or intramuscular deliveries.

In a preferred embodiment, the a adrenergic receptor agonist is delivered to a skin area subject to LTV damages by topical application on the skin,

One embodiment of the present invention relates to a method of regulating EGFR driven epithelial pathologies related to increased proliferation in a subject, which comprises topically administering to the subject a composition comprising an effective amount of an α2 adrenergic receptor. agonist and a pharmaceutically acceptable carrier.

As used herein, “regula .e” or “regulating” refers to achieving a controlled response after application of the composition.

As used herein, “EGFR” refers to Epidermal Growth Factor Receptor, the cell-surface receptor for members of the epidermal growth factor family. EGFR is a member of the ErbB family of receptors of which there are four: EGFR, also referred to as ErbB1 or HER1; ErbB2 or HER2/c-neu; Erb133 or ITER3; and Erb134 or HERA.

As used herein, an “effective amount of an a. adrenergic receptor agonist” with respect o regulating an EGFR response in a subject, means the amount of the a adrenergic receptor agonist that is sufficient to regulate EGFR response such that a disease or condition in a subject is prevented or treated.

One skilled in the art will recognize that the effective amount of the α adrenergic receptor agonist to be used in the instant invention can vary with factors, such as the particular subject to be treated, e.g., age, diet, health, etc., degree of UV radiation exposed to, severity and complications of the skin thickening sought to be treated or inhibited, the a adrenergic receptor agonist used, the formulation used, etc. In view of the present disclosure, standard procedures can be performed to evaluate the effect of the administration of a composition to a subject, thus allowing a skilled artisan to determine the effective amount of the α adrenergic receptor agonist to be administered to the subject. Such effect can be, for example, a clinically observable beneficial effect of the a adrenergic receptor agonist in reducing or inhibiting the progression of skin thickening in a subject, or an in vivo or in vitro measurement on the EGFR activity, etc.

The clinically observable beneficial effect can be a situation that, when a composition of the present invention is administered to a subject after signs and/or symptoms, such as those related to skin thickening, are observable, the signs and/or symptoms are prevented from further development or aggravation, or develop to a lesser degree than without administration of the specified composition according to embodiments of the present invention. The clinically observable beneficial effect can also be that, when a composition of the present invention is administered to a. subject before signs and/or symptoms, such as that related to skin thickening, are observable, the signs and/or symptoms are prevented from occurring or subsequently occur to a lesser degree than without administration of the composition of the present invention.

Methods of the present invention can be used in conjunction with one or more other treatments or medications for preventing or inhibiting the progression of skin or epithelium thickening, or treating existing signs and/or symptoms of skin or epithelium thickening. Examples of such other treatments or medications include, but are not limited to, retinoid and its derivatives, sun-screens or sun-blocks, anti-inflammatory agents, vitamins, such as vitamin D, nitroglycerin, etc.

Methods of the present invention can also be used in conjunction with one or more other treatments or medications for regulating an EGFR response in a subject, such as another anti-proliferative agent.

The other medicament or treatment can be administered to the subject simultaneously with, or in a sequence and within a time interval of, the administration of the α adrenergic receptor agonist, such that the active ingredients or agents can act together to treat or prevent skin thickening and signs and/or symptoms associated therewith. For example, the other medicament or treatment and the a. adrenergic receptor agonist can be administered in the same or separate formulations at the same or different times.

Any suitable route of administration can be employed to deliver the additional treatment or medication including, but not limited to, oral, intraoral, rectal, parenteral, topical, epicutaneous, transdermak subcutaneous, intramuscular, intranasal, sublingual, buccal, intradural, intraocular, intrarespiratory, or nasal inhalation.

A composition according to embodiments of the present invention comprises an effective amount or a therapeutically effective amount of an a. adrenergic receptor agonist and a pharmaceutically acceptable carrier.

The carriers useful for topical delivery of the specified compounds according to embodiments of the invention can be any carrier known in the art for topically administering pharmaceuticals, including, but not limited to, pharmaceutically acceptable solvents, such as a. polyalcohol or water; emulsions (either oil-inwater or water-in-oil emulsions), such as creams or lotions; micro emulsions; gels; ointments; liposomes; powders; and aqueous solutions or suspensions. The pharmaceutically acceptable carrier includes necessary and inert pharmaceutical excipients, including, but not limited to, binders, suspending agents, lubricants, flavorants, preservatives, dyes, and coatings.

The topical composition according to embodiments of the present invention are prepared by mixing a pharmaceutically acceptable carrier with a therapeutically effective amount of an α2 adrenergic receptor agonist according to known methods in the art, for example, methods provided by standard reference texts such as, REMINGTON: THE SCIENCE AND PRACTICE OF PHARMACY 1577-1591, 1672-1673, 866-885 (Alfonso R. Gennaro ed. 19th ed. 1995); TRANSDERMAL AND TOPICAL DRUG DELIVERY SYSTEMS (Ghosh, T. K., et al. ed. 1997), both of which are hereby incorporated herein by reference.

In one embodiment, the topical composition of the invention is in the form of an emulsion. Emulsions, such as creams and lotions are suitable topical formulations for use in the invention. An emulsion is a dispersed system comprising at least two immiscible phases, one phase dispersed in the other as droplets ranging in diameter from 0.1 μm to 100 μm. An emulsifying agent is typically included to improve stability. When water is the dispersed phase and an oil is the dispersion medium, the emulsion is termed a water-in-oil emulsion. When an oil is dispersed as droplets throughout the aqueous phase, the emulsion is termed an oil-in-water emulsion. Emulsions, such as creams and lotions that can be used as topical carriers and their preparation are disclosed in REMINGTON: THE SCIENCE AND PRACTICE OF PHARMACY 282-291 (Alfonso R. Gennaro ed. 19th ed. 1995), hereby incorporated herein by reference.

In another embodiment, the topical composition of the invention is in the form of a for example, a two-phase gel or a single-phase gel. Gels are semisolid systems consisting of suspensions of small inorganic particles or large organic molecules interpenetrated by a liquid. When the gel mass comprises a network of small discrete inorganic particles, it is classified as a two-phase gel. Single-phase gels consist of organic macromolecules distributed uniformly throughout a liquid such that no apparent boundaries exist between the dispersed macromolecules and the liquid. Suitable gels for use in the invention are disclosed in REMINGTON: THE SCIENCE AND PRACTICE OF PHARMACY 1517-1518 (Alfonso R. Gennaro ed. 19th ed. 1995), hereby incorporated herein by reference. Other suitable gels for use with the invention are disclosed in U.S. Pat. No. 6,387,383 (issued May 14, 2002); U.S. Pat. No. 6,517,847 (issued Feb. 11, 2003); and U.S. Pat. No. 6,468,989 (issued Oct. 22, 2002), each of which patents is hereby incorporated herein by reference.

In an embodiment, the topical composition further comprises an aqueous gel comprising water and a water-gelling amount of a pharmaceutically acceptable gelling agent selected from the group consisting of carbomers, glycerine polyacrylate, and mixtures thereof, and the topical composition has a physiologically acceptable pH.

As used herein, “carbomer” is the USP designat on for various polymeric acids that are dispersible but insoluble in water. When the acid dispersion is neutralized with a base a clear, stable gel is formed. Carbomer 934P is physiologically inert and is not a primary irritant or sensitizer. Other carbomers include 910, 940, 941, and 1342. Polymer thickeners (gelling agents) that may he used in compositions according to embodiments of the present invention include those known to one skilled in the art, such as hydrophilic and hydroalcoholic gelling agents frequently used in the cosmetic and pharmaceutical industries. Preferably, the hydrophilic or hydroalcoholic gelling agent comprises “CARBOPOLR®” (B.F. Goodrich, Cleveland, Ohio.), “HYPAN®” (Kingston Technologies, Dayton, N.J.), “NATROSOL®” (Aqual on, Wilmington, Del.), “KLUCELK®” (Aqualon, Wilmington, Del.), or “STABILEZE®,” (ISP Technologies, Wayne, N.J.). Preferably the gelling agent comprises between about 0.2% to about 4% by weight of the composition, More particularly, the preferred compositional weight percent range for “CARBOPOL®” is between about 0.5% to about 2%, while the preferred weight percent range for “NATROLSOL®” and “KLUCEL® is between about 0.5% to about 4%. The preferred compositional weight percent range for both “HYPAN®” and “STARILEZE®” is between 0,5% to about 4%.

“CARBOPOL®” is one of numerous cross-linked acrylic acid polymers that are given the general adopted name carbomer. These polymers dissolve in water and form a clear or slightly hazy gel upon neutralization with a caustic material such as sodium hydroxide, potassium hydroxide, triethanolamine, or other amine bases. “KLUCEL®” is a cellulose polymer that is dispersed in water and forms a uniform gel upon complete hydration, Other preferred gelling polymers include hydroxyethylcellulose, cellulose gum, MVEIMA decadiene crosspolymer, PVM/MA copolymer, or a combination thereof.

In another preferred embodiment, the topical composition of the invention is in the form of an ointment. Ointments are oleaginous semisolids that contain little if any water. Preferably, the ointment is hydrocarbon based, such as a wax, petrolatum, or gelled mineral oil. Suitable ointments for use in the invention are well known in the art and are disclosed in REMINGTON: THE

SCIENCE AND PRACTICE OF PHARMACY 1585-1591 fonso R. Gennaro ed. 19th ed, 1995), hereby incorporated herein by reference.

in an embodiment of the present invention, the topical composition of the invention comprises at least one of a cream and an ointment, each comprising an agent selected from the group consisting of stearic acid, stearyl alcohol, cetyl alcohol, glycerin, water, and mixtures thereof, and the topical composition has a physiologically acceptable pH.

In another embodiment, the topical composition of the invention is in the form of an aqueous solution or suspension, preferably, an aqueous solution. Suitable aqueous topical formulations for use in the invention include those disclosed in REMINGTON: THE SCIENCE AND PRACTICE OF PHARMACY 1563-1576 ‘Alfonso R. Gennaro ed. 19th ed. 1995), hereby incorporated herein by reference. Other suitable aqueous topical carrier systems include those disclosed in U.S. Pat, No. 5,424,078 (issued Jun. 13, 1995); U.S. Pat. No. 5,736,165 (issued Apr. 7, 1998); U.S. Pat. No. 6,194,415 (issued Feb. 27, 2001); U.S. Pat. No. 6,248,741 (issued Jun. 19, 2001); and U.S. Pat. No. 6,465,464 (issued Oct. 15, 2002), all of which patents are hereby incorporated herein by reference.

The pH of the topical formulations of the invention are preferably within a physiologically acceptable pH, e.g., within the range of about 5 to about 8, more preferably, of about ’.5 to about 6.5. To stabilize the pH, preferably, an effective amount of a buffer is included. In one embodiment, the buffering agent is present in the aqueous topical formulation in an amount of from about 0.05 to about 1 weight percent of the formulation. Acids or bases can be used to adjust the pH as needed.

Tonicity-adjusting agents can be included in the aqueous topical formulations of the invention. Examples of suitable tonicity-adjusting agents include, but are not limited to, sodium chloride, potassium chloride, mannitol, dextrose, glycerin, and propylene glycol. The amount of the tonicity agent can vary widely depending on the formulation's desired properties. In one embodiment, the tonicity-adjusting agent is present in the aqueous topical formulation in an amount of from about 0.5 to about 0.9 weight percent of the formulation.

Preferably, the aqueous topical formulations of the invention have a viscosity in the range of from about 15 cps to about 25 cps. The viscosity of aqueous solutions of the invention can be adjusted by adding viscosity adjusting agents, for example, but not limited to, polyvinyl alcohol, povidone, hydroxyprom4 methyl cellulose, poloxamers, carboxymethyl cellulose, or hydroxyethyl cellulose.

In a preferred embodiment, the aqueous topical formulation of the invention is isotonic saline comprising a preservative, such as benzalkonium chloride or chlorine dioxide, a viscosity-adjusting agent, such as polyvinyl alcohol, and a buffer system such as sodium citrate and citric acid.

The topical composition according to embodiments of the invention can comprise pharmaceutically acceptable excipients such as those listed in REMINGTON: THE SCIENCE AND PRACTICE OF PHARMACY 866-885(Alfonso R. Gennaro ed. 19th ed. 1995); and TRANSDERMAL AND TOPICAL DRUG DELIVERY SYSTEMS (Ghosh, T. K. et al. (.1. 1997), hereby incorporated herein by reference, including, but not limited to, protectives, adsorbents, demulcents, emollients, preservatives, antioxidants, moisturizers, buffering agents, solubilizing agents, skin-penetration agents, and surfactants.

In an embodiment, the topical composition of the invention further comprises one or more agents selected from the group consisting of a preservative, a local anesthetic and a skin humectant.

Suitable preservatives include, but are not limited to, quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; alcoholic agents, for example, chlorobutanol, phenylethyl alcohol, and benzyl alcohol; antibacterial esters, for example, esters of parahydroxybenzoic acid; and other anti-microbial agents such as chlorhexidine, chlorocresol, benzoic acid and polymyxin.

The topical composition according to embodiments of the invention can include pharmaceuticals or their pharmaceutically acceptable salts, such as an α2 adrenergic receptor agonist, and optionally one or more other pharmaceutically active ingredients, including, but not limited to, corticosteroids and other anti-inflammatory agents, such as betamethasone, ditlorasone, amcinonide, fluocinolone, mometasone, hydrocortisone, prednisone, and triamcinolone; local anesthetics and analgesics, such as camphor, menthol, lidocaine, and dibucaine, and pramoxine; antifungals, such as ciclopirox, chloroxylenol, triacetin, sulconazole, nystatin, undecylenic acid, tolnaftate, miconizole, clotrimazole, oxiconazole, griseofulvin, econazole, ketoconozole, and amphotericin B; antibiotics and anti-infectives, such as mupirocin, erythromycin, clindamycin, gentamicin, polymyxin, bacitracin, and silver sulfadiazine; and antiseptics, such as iodine, povidine-iodine, benzalkoniutn chloride, benzoic acid, chlorhexidine, nitrofurazine, benzoyl peroxide, hydrogen peroxide, hexachlorophene, phenol, resorcinol, and cetylpyridinium chloride.

In a preferred embodiment, a topical composition according to embodiments of the invention further comprises titanium dioxide (TiO₂), preferably at an amount that is sufficient to mask the color of brimonidine or another colored ingredient in the formulation, but would not cause irritation to the skin. TiO₂ may cause mild irritation and reddening to the eyes, thus eye contact with the TiO₂containing topically administrable composition should be avoided.

Dosages and dosing frequency will be determined by a trained medical professional depending on the activity of the compounds used, the characteristics of the particular topical formulation, and the identity and severity of the dermatologic disorder treated or prevented.

in an embodiment of the present invention, the topical composition comprises 0.01% to 5% by weight of an a adrenergic receptor agonist, such as an α2 adrenergic receptor agonist. For example, the composition can comprise, 0.01%, 0.05%, 0.1%, 0.2%, 0.3%, 0.4%, 0.5%, 0.6%, 0.7%, 0.8%, 0,9%, 1%, 2%, 3%, 4% or 5%, by weight, of the a2 adrenergic receptor agonist.

To prevent or inhibit skin thickening in view of the present disclosure, for example, the topical compositions of the invention are topically applied directly to the area exposed to sunlight or the otherwise affected area in any conventional manner well known in the art, e.g., by dropper or applicator stick, as a mist via an aerosol applicator, via an intradermal or transdermal patch, or by simply spreading a formulation of the invention onto the affected area with fingers. Generally the amount of a topical formulation of the invention applied to the affected skin area ranges from about 0.1 g/cm² of skin surface area to about 5 g/cm², preferably, 0.2 g/cm² to about 0.5 g/cm² of skin surface area. Typically, one to four applications per day are recommended during the term of treatment.

The topical formulations of the invention can be filled and packaged into a plastic squeeze bottle or tube. Suitable container-closure systems for packaging a topical formulation of the invention are commercially available for example, from Wheaton Plastic Products, 1101 Wheaton Avenue, Millville, N.J. 08332.

Preferably, instructions are packaged with the formulations of the invention, for example, a pamphlet or package label. The labeling instructions explain how to administer topical formulations of the invention, in an amount and for a period of time sufficient to prevent or inhibit skin thickening and signs and/or symptoms associated therewith. Preferably, the label includes the pharmacology, drug resistance, pharmacokinetics, absorption, bioavailability, and contraindications.

This invention will be better understood by reference to the non-limiting examples that follow, but those skilled in the art will readily appreciate that the examples are only illustrative of the invention as described more fully in the claims which follow thereafter.

### EXAMPLE 1

### Aqueous Topical Formulations

This example illustrates aqueous topical formulations that can be used in the present invention.

A first aqueous solution topical formulation comprises: brimonidine tartrate (0.01% to 5% w/w); Puriteg (0.005% w/w) (stabilized chlorine dioxide) as a preservative; and the inactive ingredients: boric acid; calcium chloride; magnesium chloride; potassium chloride; purified water; sodium borate; sodium carboxymethylcellulose; sodium chloride; with hydrochloric acid and/or sodium hydroxide to adjust the pH to 5.6 to 6.6. The osmolality is in the range of 250-350 mOstnol/kg.

A second aqueous solution topical formulation comprises brimonidine tartrate (0.2% to 2% w/w); benzalkonium chloride (0.005% w/w.) as a preservative; and the inactive ingredients: boric acid; calcium chloride; magnesium chloride; potassium chloride; purified water; sodium borate; sodium carboxymethylcellulose; sodium chloride; with hydrochloric acid and/or sodium hydroxide to adjust the pH to 5.6 to 6.6, The osmolality is in the range of 250-350 mOsmol/kg.

### EXAMPLE 2

### Cream or Ointment Topical Formulations

This example illustrates cream or ointment topical formulations that can be used in the present invention,

A first cream topical formulation (hydrophilic ointment) is described in Table 2 below.

TABLE 2

| Ingredient | Weight Percent |
|---|---|
| Brimonidine tartrate | 0.01% to 5% |
| Stearic acid | 7% |
| Stearyl alcohol | 5% Cetyl alcohol |
| 2% Glycerin | 10% |
| Sodium lauryl sulfate | 1% |
| Propylparaben | 0.05% |
| Methylparaben | 0.25% |
| Disodium edetate | 0.055% |
| Distilled water | QS |
| TOTAL | 100% |

To Whom It May Concern: make the formulation, the stearyl alcohol and the white petrolatum are melted on a steam bath, and warmed to about 75 degrees C. The other ingredients, previously dissolved in the water and warmed to 75 degrees C., are then added, and the mixture is stirred until it congeals. The mixture is then allowed to cool with stirring, and brimonidine tartrate is then added as a concentrated solution.

An ointment topical formulation (hydrophilic ointment) is described in Table 3 below.

TABLE 3

| Ingredients | Weight |
|---|---|
| Brimonidine tartrate | 20 g |
| Cholesterol | 30 g |
| Stearyl Alcohol | 30 g |
| White Wax | 80 g |
| White Petrolatum | 820-800 g |

To make the formulation, the stearyl alcohol and white wax are mixed together on a steam bath. The cholesterol is then added and stirred until it completely dissolved. The white petrolatum is then added and mixed. The mixture is removed from the bath, and stirred until it congeals. With continuous stirring, ‘brimonidine tartrate is added as a concentrated slurry.

### EXAMPLE 3

### Gel Topical Formulations

This example illustrates gel topical formulations that can be used in the present invention.

A first gel formulation is described in Table 4 below.

TABLE 4

| Ingredients | Weight % |
|---|---|
| Brimonidine tartrate | 0.01-5% |
| Methylparaben NF | 0.15% |
| Propylparaben NF | 0.03% |
| Hydroxyethylcellulose NF | 1.25% |
| Disodium Edetate USP | 0.05% |
| Purified Water, USP | QS |
| TOTAL | 100% |

A second gel formulation is described in Table 5 below.

TABLE 5

| Ingredients | Weight % |
|---|---|
| Brimonidine tartrate | 0.5% |
| Methylparaben | 0.20% |
| Propylparaben | 0.05% |
| Carbomer 934P NF | 1.0% |
| Sodium Hydroxide | QS pH 7 |
| Purified Water, USP | QS |
| TOTAL | 100% |

The ingredients are mixed together and aqueous sodium hydroxide is slowly added to the mixture until a pH of about 7 is reached and the gel is formed.

A third gel formulation is described in Table 6 below.

TABLE 6

| Ingredient | Weight Percent |
|---|---|
| Brimonidine tartrate | 0.1-2% |
| Carbomer 934P | 1.25% |
| Methylparaben | 0.3% |
| Phenoxyethanol | 0.4% |
| Glycerin | 5.5% |
| 10% Titanium dioxide | 0.625% |
| Propylene glycol | 5.5% |
| 10% NaOH Solution | 6.5% |
| DI Water | QS |
| TOTAL | 100% |

A fourth gel formulation is described in Table 7 below.

TABLE 7

| Ingredients | Weight % |
|---|---|
| Brimonidine tartrate | 0.01-5% |
| Methylparaben | 0.2% |
| Propylparaben | 0.05% |
| “CARBOPOL ®” | 1.0% |
| Triethanolamine | QS pH 7 |
| Water | QS |
| TOTAL | 100% |

The ingredients are mixed together and stirred. Triethanolamine is added until a pH of about 7 is attained.

### EXAMPLE 4

### Foam Topical Formulations

This example illustrates foam topical formulations that can be used in the present invention.

A first foam formulation is described in Table 8 below.

TABLE 8

| Ingredients | Amount (Weight %) |
|---|---|
| Brimonidine tartrate | 0.01-5 |
| Stearic Acid | 4.2 |
| Laureth-23 | 1.4 |
| Sodium Lauryl Sulfate | 0.5 |
| Triethanolamine | 2.2 |
| Butylated hydroxytoluene (BHT) | 0.01 |
| Fragrance | 0.5 |
| Aeron A-31 Propellant | 3 |
| Water | QS |
| TOTAL | 100 |

The water is heated to 80-85° C., after which stearic acid is added. Once the stearic acid is melted, the la.ureth-23 is added, melted, and mixed well. Next, triethanolamine is added and the resulting composition is mixed well for about 30 minutes to form a soap. The resulting soap is then cooled to about 65° C., after which sodium lauryl sulfate is added. The composition is then mixed well. Next, the BHT and the Brimonidine tartrate are added, followed by mixing. The resulting composition is then cooled to room temperature and the fragrance added. The product is packaged with the Aeron A-31 propellant in an aerosol can using conventional techniques and mechanically shaken for 5 minutes. The product dispenses as a cone-shaped spray that deposits onto the skin as a layer of rich lather that quickly covers a wide area of skin, and begins to relieve symptoms within about 2 minutes after application.

A second foam formulation is described in Table 9 below.

TABLE 9

| Ingredient | Amount (Weight %) |
|---|---|
| Brimonidine tartrate | 0.2-2 |
| Water | QS |
| Palmitic Acid | 2.12 |
| Laureth-23 | 0.93 |
| Triethanolamine (99%) | 1.13 |
| Cetyl Dimethicone Copolyol | 0.19 |
| Mineral Oil | 0.31 |
| Stearyl Alcohol | 0.31 |
| Lauramide DEA | 0.15 |
| PEG-150 Distearate | 0.05 |
| Imidazolidinyl Urea | 0.0016 |
| Methylparaben | 0.0005 |
| Propylparaben | 0.00003 |
| Freeze Dried Aloe Powder | 0.0015 |
| Fragrance | 0.50 |
| Aeron A-31 Propellant | 3.00 |
| TOTAL | 100 |

The aqueous phase is prepared as follows. The water is heated to 80° C., after which palmitic acid is added. Once the palmitic acid is melted, the laureth-23 is added, melted, and mixed well. Next, triethanolamine is added and the resulting composition is mixed well for about 15 minutes to form a soap.

Stearyl alcohol, mineral oil, lauramide DEA, cetyl dimethicone copolyol, PEG-1 50 distearate, and BHT are mixed and heated at 55° C. to form the oil phase. The oil phase is combined with the aqueous phase at 80° C. and mixed well for about 15 minutes. The resulting mixture is then cooled to room temperature and the imidazolidinyl urea, methylparaben, and propylparaben are added, and then mixed well. The brimonidine tartrate is then added, and mixed well. Next, the fragrance is added, followed by gentle mixing. The aloe is then dissolved in make-up water and added with slow mixing to form the product formulation which is then packaged in an aerosol can as described for the first foam formulation.

The product dispenses as a cone-shaped spray that deposits onto the skin as a layer of rich lather that quickly covers a wide area of skin, and begins to relieve symptoms within about 2 minutes after application.

A third non-soapy foam formulation is described in Table 10 below.

TABLE 10

| Ingredient | Amount (Weight %) |
|---|---|
| Brimonidine tartrate | 0.4-0.6 |
| Ethanol | 6 |
| Ethyl Ester of PVM/MA | 4 |
| Copolymer Dimethicone Copolyol | 0.1 |
| Water | QS |
| PVP/VA Copolymer | 1 |
| Sodium Lauryl Sulfate | 1 |
| Oleth-20 | 0.5 |
| Cocamide MEA | 0.05 |
| Methyl Paraben | 0.1 |
| Aminomethyl Propanol | 0.53 |
| Stearalkonium Chloride | 0.05 |
| Steareth-16 | 0.1 |
| Panthenol | 0.5 |
| Fragrance | 0.5 |
| Aeron A-46 | 5 |
| TOTAL | 100 |

The alcohol phase is prepared by dissolving ethyl ester of PVM/MA copolymer in ethanol, after which dimethicone is added and mixed well. The aqueous phase is prepared by heating the water to 65° C., after which the PVP/VA copolymer is added and mixed well. The oil phase is prepared by mixing the oleth-20, cocamide MEA, and steareth-16 at 60° C. to form a blend. The oil phase is then added to the aqueous phase at 65° C. and mixed well. Next, the methylparaben is added to the mixture, followed by mixing, after which the aminomethyl propanol, stearalkonium chloride, and panthenol are added and mixed until uniform. The resulting composition is cooled to room temperature, after which the alcohol phase is added and mixed well. The fragrance is then added and mixed gently to form the product. The product is then packaged in an aerosol can.

The product dispenses as a cone-shaped spray that deposits onto the skin as a layer of rich lather that quickly covers a wide area of skin, and begins to relieve symptoms within about 2 minutes after application.

### EXAMPLE 5

### Photo Study Brimonidine

Albino hairless SKHI-hr mice (36/sex/group) were treated for 40 weeks with UVR and brimonidine gel or vehicle according to the design in Table 11. Mice were further observed for 12 weeks without treatment. Topical treatments were performed approximately one hour before UVR on Monday, Wednesday and Friday of each week and approximately one hour after UVR Tuesday and Thursday of each week. See Table 11.

All procedures involving animals were conducted in a fully accredited animal facility and in accordance with the preapproved protocols.

TABLE 11

| | | | | | | Treatment |
|---|---|---|---|---|---|---|
| | | | Frequency of | Solar- | Duration of | free |
| | Brimonidine | Administration | administration | simulated | Treatment | follow-up |
| Dosage | tartrate | (μL/mouse, on | (days per | UVR dose | or exposure | period |
| Group | Conc. (%) | 25 cm² BSA) | wk)* | (RBU/week) | (weeks) | (weeks) |
| 1 | Vehicle | 100 | 5 | 600 | 40 | 12 |
| 2 | 0.18 | 100 | 5 | 600 | 40 | 12 |
| 3 | 1 | 100 | 5 | 600 | 40 | 12 |
| 4 | 2 | 100 | 5 | 600 | 40 | 12 |
| 5 | N/A | N/A | N/A | 600 | 40 | 12 |
| 6 | N/A | N/A | N/A | 1200 | 40 | 12 |

N/A: NOT APPLICABLE
BSA: body surface area
RBU: Robertson-Berger Unit (a measure of effectiveness of UVR; 400 RBU approximates one minimal erythema dose in previously untanned human skin)
*Monday, Wednesday and Friday of each week: exposure to UVR approximately one hour after test item application. Tuesday and Thursday of each week: exposure to UVR approximately one hour before test item application.

As the results shown in Table 12, topical application of brimonidine at 0.18%. 1%, and 2% (w/w) concentrations surprisingly resulted in a dose-dependent reduction in UV-induced skin thickening, The UVR, exposure was 600 RBU/week for all test groups in the table.

TABLE 12
Group Comparisons of Skin Thickening Prevalence

| Group | Vehicle | 0.18% | 1% | 2% | UV control |
|---|---|---|---|---|---|
| UV treatment | Yes | Yes | Yes | Yes | Yes |
| Male tested | 36 | 36 | 36 | 36 | 36 |
| Skin | 589/35 | 514/34 | 392/27** | 367/24** | 797/35 |
| thickening | | | | | |
| Female tested | 36 | 36 | 36 | 36 | 36 |
| Skin | 554/33 | 521/32 | 374/25** | 381/23** | 577/34 |
| thickening | | | | | |

**p < 0.01 compared to the vehicle control group

N/N=Total number of observations/number of mice with observation

Although treatment with 0.18% (w/w) brimonidine tartrate did not statistically reduce the UV-induced skin thickness, the incidence of skin thickening in this treatment group was still observably lower than that in the UV control group. Both groups treatment with 1% and 2% (w/w) brimonidine tartrate statistically reduced the UV-induced skin thickness compared to the UV control group. The observed reduction was clearly related to brimonidine as the vehicle control group has no effect compared to the UV control group alone, indicating that an alpha adrenergic receptor agonist is effective in reducing skin thickening, such as that induced by UV.

In addition to skin thickening, other skin disorders induced by UV-irradiation, such as low grade of erythema or flaking, white raised area on skin, or wrinkling, were also reduced in groups administered with 1% or 2% w/w) brimonidine tartrate, as compared to the group administered with vehicle control,

While not wishing to be bound by theory, the observed reduction in the UV-induced skin thickening appeared to be due, at least in part, to the regulation of the EGFR response, e.g., by inhibiting the EGFR related keratinocyte proliferation or EGFR related suppression of apoptosis when skin is stimulated by UV radiation. Thus, an alpha adrenergic receptor agonist can be used to regulate an EGFR response for the treatment of a disease or disorder associated with EGFR.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A method of reducing or inhibiting the progression of a skin thickening in a subject in need thereof, comprising topically administering to a skin area of the subject a topical composition comprising an effective amount of at least one alpha adrenergic receptor agonist and a pharmaceutically acceptable carrier, wherein the skin area has, or is prone to have, the skin thickening, provided that the skin thickening is not associated with one or more selected from the group consisting of a skin tumor, rosacea, erythema, telangiectasias, psoriasis, purpura, sagging skin and wrinkle.

2. The method of claim 1, wherein the topical composition is selected from the group consisting of an aqueous solution topical formulation, a topical gel formulation, a cream topical formulation, and an ointment formulation.

3. The method of claim 1, wherein the skin thickening is associated with one or more conditions selected from the group consisting of sun exposure, hormonal imbalance, a deficiency in vitamin and/or antioxidant, epidermal hyperplasia, keratinocyte proliferation, EGFR-dependent cell division, and combinations thereof.

4. The method of claim 1, wherein the skin thickening is associated with one or more conditions selected from the group consisting of CREST syndrome, corns and calluses, warts, hives, keratosis, atopic dermatitis, eczema, scleroderma, lipoderamtoscelerosis, age spots (or lentigo).

5. The method of claim 1, wherein the skin thickening is induced by an UV-irradiation.

6. The method of claim 1, wherein the at least one alpha-adrenergic receptor agonist is an agonist selective for an alpha 2-adrenergic receptor.

7. (canceled)

8. (canceled)

9. The method according to claim 6, wherein the at least one alpha-adrenergic receptor agonist is brimonidine, and the composition comprises from about 0.1% to about 2% by weight of brimonidine.

10. The method of claim 1, wherein the at least one alpha-adrenergic receptor agonist is selected from the group consisting of (8-Bromo-quinoxalin-6-yl)-(4,5-dihydro-1H-imidazol-2-yl)-amine, (8-Bromo-quinoxalin-5-yl)-(4,5-dihydro-1H-imidazol-2-yl)-amine, (5-B romo-3-methyl-qu inoxal in-6-yl)-(4,5-dihydro-1H-im idazol-2-yl)-am ine, (5-Bromo-2-methoxy-quinoxalin-6-yl)-(4,5-dihydro-1H-im idazol-2-yl)-amine, (4,5-dihydro-1H-imidazol-2-yl)-(8-methyl-quinoxalin-6-yl)-amine, (4,5-dihydro-1H-imidazol-2-yl)-quinoxalin-5-yl-amine, naphazoline, tetrahydrozoline, oxymetazoline, xylometazoline, epinephrine, norepinephrine, phenylephrine, methoxamine, mephentermine, metaraminol, and midodrine.

11. The method of claim 1, further comprising administering to the subject at least one additional agent useful for reducing or inhibiting the progression of skin thickening.

12. The method of claim 11, wherein the addition agent is selected from the group consisting of retinoid and derivatives thereof, sun screens, sun-blocks, anti-inflammatory agents, vitamins and nitroglycerin.

13. (canceled)

14. (canceled)

15. The method of claim 1, wherein the topical composition is administered to the skin area twice daily.

16. A method of inhibiting a skin disorder induced by an UV-irradiation in a subject in need thereof, comprising topically administering to a skin area of the subject a topical composition comprising an effective amount of at least one alpha adrenergic receptor agonist and a pharmaceutically acceptable carrier.

17. The method of claim 16, wherein the skin disorder is grade 1 erythema, grade 1 flaking, wrinkling, white raised area on skin, or skin thickening induced by sun exposure.

18. The method of claim 16, wherein the alpha adrenergic receptor agonist is brimonidine.

19. A method of regulating an EGFR response in a subject in need thereof to thereby result in treating or preventing a disease or condition associated with EGFR in the subject, comprising administering to the subject a composition comprising an effective amount of an a adrenergic receptor agonist and a pharmaceutically acceptable carrier, provided that the disease or condition associated with EGFR is not associated with one or more selected from the group consisting of a skin tumor, rosacea, erythema, telangiectasias psoriasis, purpura, sagging skin and wrinkle.

20. The method of claim 19, wherein the EGFR response is induced by an UV-irradiation.

21. The method of any of claim 19, wherein the composition is administered to the subject topically.

22. The method according to claim 19, wherein the a adrenergic receptor agonist is brimonidine.

23. (canceled)

24. The method according to claim 22, wherein the composition comprises about 0.1% to about 2% by weight of the a2 adrenergic receptor agonist.

25. The method according to claim 19, further comprising administering to the subject at least one additional agent useful for treating or preventing the disease or condition in the subject.
